(19) United States
(12) Patent Application Publication
(10) Pub. No.: US 2016/0287485 A1
(43) Pub. Date: Oct. 6, 2016

(54) AGENT AND METHOD FOR THE TEMPORARY DEFORMATION OF KERATIN FIBRES

(71) Applicant: Henkel AG & Co. KGaA, Duesseldorf (DE)

(72) Inventors: Anna Puls, Winsen (DE); Jisook Baek, Shanghai (CN)

(73) Assignee: Henkel AG & Co. KGaA, Duesseldorf (DE)

(21) Appl. No.: 15/186,243

(22) Filed: Jun. 17, 2016

Related U.S. Application Data
(63) Continuation of application No. PCT/DE2014/200536, filed on Oct. 8, 2014.

(30) Foreign Application Priority Data
Dec. 23, 2013 (DE) 10 2013 227 117.4

(51) Int. Cl. A61K 8/02; A61K 8/25; A61K 8/81; A61Q 5/06

(52) U.S. Cl. CPC A61K 8/022; A61Q 5/06; A61K 8/25; A61K 8/8158

(57) ABSTRACT

The invention relates to powdered cosmetic compositions including a) 10 to 30 wt. % hydrophobized metal oxide power, b) 10 to 89 wt. % organic polyol, c) 0.01 to 15 wt. % of at least one copolymer of the monomers b1) N-tert-octylacrylamide, b2) acrylic acid, b3) tert-butylaminoethyl methacryclic acid, and b4) possibly additional monomers, which compositions are especially suitable for deforming keratin fibers and provide shine, texture and silkiness.

### FIELD OF THE INVENTION

The present invention generally relates to the technical field of the temporary deformation of keratin-containing fibers, in particular human hair.

### BACKGROUND OF THE INVENTION

Styling agents for deforming keratin-containing fibers have long been known and are used in various forms to construct, revive and fix hairstyles which, in many hair types, can only be achieved using stabilizing active ingredients. In this case, hair treatment agents that are used to shape the hair both permanently and temporarily play a key part. Temporary shapes, which should provide good hold without affecting the healthy appearance of the hair, for example the shine thereof, can be achieved for example by means of hair sprays, hair waxes, hair gels, hairdryers, etc.

Corresponding agents for temporary shaping typically include synthetic polymers as the shaping component. Preparations including a polymer can be applied to the hair by means of propellants or by a pump mechanism. On the other hand, hair gels and hair waxes are generally not applied directly to the hair, but instead are distributed through the hair by means of a comb or the hands.

Known forms of temporary styling agents can often not be dispensed with sufficient accuracy. For example, as soon as they are applied to the hair, hair gels, hair creams and hair waxes can only be distributed with difficulty. Hair sprays can be distributed onto the hair more uniformly. However, since the user cannot visually determine the total amount of styling agent applied, there is the risk that more styling agent will be applied to the hair than is actually necessary.

Powdered cosmetic agents are known and have already been used for a long time in the field of skin treatment, for example. Typical examples are, for example, make-up powder or eye shadow. To obtain the powdery consistency, a powdered carrier material can be used. A metal oxide, for example, such as silicon dioxide, can be used as a suitable carrier material. Hydrophobized metal oxide or silicon dioxide is of particular interest. This can be obtained for example from pyrogenic silicon dioxide, which can be obtained commercially in various specifications. Untreated pyrogenic silicon dioxide carries silanol groups and siloxane groups on the surface. Therefore, it has a high affinity for water, i.e. it is hydrophilic. By reacting it with suitable organic silicon compounds, alkyl silyl groups can chemically bond on the surface of the pyrogenic silicon dioxide. This produces modified silicon dioxide powders that can no longer be wetted by water, i.e. have hydrophobic properties. These hydrophobized silicon dioxides are suitable for producing dry water, in which the drops of water are prevented from flowing back together again. The resultant powders can have a water content of up to 95%. Upon mechanical loading, for example when rubbed on the skin, the enclosed water is released again.

Liquefiable cosmetic or pharmaceutical powder compositions are described, for example, in EP 1235554 B1. The international application WO 03/037287 A1 discloses the use of a granulate based on pyrogenic silicon dioxide in cosmetic compositions. The special granulates can be silanized, i.e. hydrophobized, and are suitable for producing cosmetic compositions of all consistencies, for example liquids, foams, sprays or powders. The international application WO 2007/051511 A1 describes the use of a powdered composition having 50 to 95 wt. % of an aqueous solvent, hydrophobized silicon dioxide powder, and at least one film-forming and/or stabilizing polymer in the aqueous solvent for temporarily deforming keratin fibers. The German patent application DE 102008057261 A1 relates to powdered compositions that are used to temporarily reshape hair to create a very strong hold for the fixed hairstyle.

Although the powdered hair cosmetic agents of the prior art provide acceptable levels of hold for hair reshaping and are distinguished on account of being easy to dispense, the result achieved by these agents as regards the parameters of natural shine, elasticity and hold can still be improved just as much as their ease of application, for example by rubbing on the hands and hair. In addition, most of the common styling starting materials such as waxes, oils or polymers are not readily suitable for producing stable powdered compositions. They either prevent the successful formation of core-shell particles, or the storage stability of the core-shell particles formed is reduced.

The object of the present invention was therefore to provide powdered hair treatment agents for temporary shaping that are stable in storage, can be precisely and simply dispensed and applied, do not clump the hair, and provide the hair with a fuller and natural hold and natural shine, while not affecting the durability of the styling result in the process.

Furthermore, other desirable features and characteristics of the present invention will become apparent from the subsequent detailed description of the invention and the appended claims, taken in conjunction with this background of the invention.

### BRIEF SUMMARY OF THE INVENTION

Powdered cosmetic compositions including 10 to 30 wt. % of a hydrophobized metal oxide powder, 10 to 89 wt. % of an organic polyol, and 0.01 to 15 wt. % of at least one copolymer from the monomers: N-tert-octylacrylamide, acrylic acid, tert-butylaminoethyl methacrylic acid, and possibly other monomers.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has been found that powdered cosmetic compositions on the basis of a polyolemulsifier mixture achieve the above object. The present application first relates to powdered cosmetic compositions including
a) 10 to 30 wt. % of hydrophobized metal oxide powder
b) 10 to 89 wt. % of organic polyol
c) 0.01 to 15 wt. % of at least one copolymer consisting of the monomers
   b1) N-tert-octylacrylamide
   b2) acrylic acid
   b3) tert-butylaminoethyl methacrylic acid
   b4) and possibly other monomers.

The powdered compositions according to the invention are preferably in the form of core-shell particles, the shell of which includes particles of at least one hydrophobized metal oxide powder and the liquid core of which includes organic polyol and possibly water. Within the context of the invention, particles are particles (cf. DIN 66160: 1992-09) of solids present in the form of grains. Within the context of the invention, powdered means compositions of which the particles are free-flowing under their own weight (cf. DIN EN ISO 6186: 1998-08).

The powdered compositions according to the invention are characterized in that the liquid core is released from the core-shell particles by mechanically loading the core-shell particles, in particular by rubbing and/or pressure, and in the process a liquid is formed from the powdered composition. The composition is thus a powdered, powder-to-liquid composition. The powdered compositions according to the invention can be dispensed very easily. They can also be distributed very evenly through the hair since the liquid core is not released until mechanical loading is applied at the site of action and the hair fibers can be wetted in a targeted manner. Therefore, the powder can first be carefully distributed through the hair and only then is greater mechanical loading applied thereto, for example by massaging the powder into the hair in a targeted manner. As a result, the styling effect only develops directly on the desired portion of the hair.

The powdered compositions used include hydrophobized metal oxide. Preferred compositions are characterized in that they include, based on their total weight, the hydrophobized metal oxide powder in amounts of 10 to 25, preferably 12 to 22 wt. %. In each case, the optimum amount is determined in particular by the hydrophobicity of the silicon dioxide powder used. The more hydrophobic the silicon dioxide powder, the less is needed thereof to obtain a stable powdered product.

In principle, provided that a powdered product is produced when the metal oxide is intensively mixed with the liquid, aqueous phase, there is no restriction on the type of hydrophobized metal oxide. Within the context of the invention, hydrophobized is understood to mean those metal oxides that have been modified at least on the surface of the particle in such a way that the modified particle is wetted by water to a lesser extent than the unmodified particle. In particular, silanized, hydrophobized metal oxides are preferred. According to the invention, at least one representative from the group formed by silanes, halogen silanes, alkoxy silanes and silazanes is preferably suitable as the reagent for silanizing the metal oxide. Preferably suitable hydrophobized metal oxides of the hydrophobized metal oxide powder are selected according to the invention from at least one representative of the group formed by hydrophobized silicates, hydrophobized aluminum silicates, hydrophobized titanium dioxide and hydrophobized silicon dioxide. Hydrophobized silicates have proven particularly suitable for producing the cosmetic compositions according to the invention, pyrogenic silicic acid post-treated by silanization or by reaction with polydimethylsiloxane being particularly advantageous.

The powdered compositions used preferably include hydrophobized silicon dioxide. In principle, provided that a powdered product is always produced when the hydrophobized silicon dioxide is intensively mixed with the polyol b), the copolymer c) and possible other ingredients, there is no restriction on the type of hydrophobized silicon dioxide.

Particularly preferably, the powdered composition according to the invention includes at least silanized, hydrophobized silicon dioxide as the hydrophobized metal oxide powder. According to the invention, at least one representative of the group formed by silanes, halogen silanes, alkoxy silanes and silazanes is preferably suitable as the reagent for silanizing the silicon dioxide. Preferred representatives of the group of silanes are hexa(C₁-C₂₀)alkyldisilane, in particular hexamethyldisilane.

If a halogen silane is used as a silylating agent, at least one compound from the group formed by the following compounds is selected as the preferred halogen silane:

[(C₁-C₂₀)alkyl]_{z′}SiX_{(4-z′)}

X₃Si[(CH₂)ₙ-R]

X₂[(C₁-C₂₀)alkyl]Si(CH₂)ₙ-R

[(C₁-C₂₀)alkyl]_{(y′+1)}[R—(CH₂)ₙ]_{(2-y′)}SiX

where
X represents a chlorine, bromine or iodine atom,
z′ is 1, 2 or 3,
y′ is 0, 1 or 2,
n is an integer from 1 to 20, and
R stands for a functional group from
(C₁-C₁₀) alkyl—, aryl—, (C₁-C₆) perfluoroalkyl—, —NH₂, —N₃, —SCN, —CH═CH₂, —O(O)C—C(CH₃)═CH₂, —OCH₂—CH═CH₂,

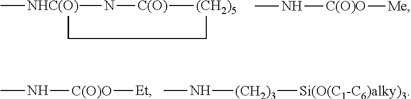

If an alkoxysilane is used as a silylating agent, at least one compound from the group formed by the following compounds is selected as the preferred alkoxysilane:

[(C₁-C₂₀)alkylO]_{z}Si(C₁-C₂₀)alkyl_{(4-z)}

[(C₁-C₂₀)alkylO]_{z}Si[(CH2)ₙ-R]_{(4-z)}

[(C₁-C₂₀)alkylO]₂[(C₁-C₂₀)alkyl]Si(CH₂)ₙ-R

[(C₁-C₂₀)alkylO][(C₁-C₂₀)alkyl]₂Si(CH₂)ₙ-R

[(C₁-C₂₀)alkylO][(C₁-C₂₀)alkyl]Si[(CH₂)ₙ-R]₂

(C₁-C₂₀alkyl)₃SiO—C(CH₃)═N—Si(C₁-C₂₀)alkyl₃,

where
n is an integer from 1 to 20 and
z represents the number 1, 2 or 3
R stands for a functional group from
(C₁-C₂₀)alkyl—, aryl—, (C₁-C₆)perfluoroalkyl—, —NH₂, —N₃, —SCN, —CH═CH₂, —O(O)C—C(CH₃)═CH₂, —OCH₂—CH═CH₂,

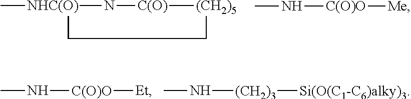

As a preferred silazane, at least one compound from the class of disilazanes is selected, in particular at least one compound selected from disilazanes of the formula

R′₂R″Si—NH—SiR′₂R″

where
R′ represents a (C₁-C₂₀) alkyl group and
R″ represents a (C₁-C₂₀) alkyl group or a vinyl group. A particularly preferred silazane is hexamethyldisilazane.

All of the above-mentioned alkyl groups, whether (C₁-C₆) alkyl, (C₁-C₁₀) alkyl, or (C₁-C₂₀) alkyl, can be both cyclic and linear or branched. Examples of alkyl groups that can be used according to the invention are methyl, ethyl, n-propyl, isopropyl, n-butyl, cyclopentyl, cyclohexyl, n-decyl, lauryl, myristyl, cetyl, stearyl, isostearyl and behenyl. An example of an aryl group according to the invention is the phenyl group. Examples of a (C₁-C₆) perfluoroalkyl group according to the invention are trifluoromethyl, perfluoroethyl, perfluoropropyl and perfluorohexyl. Preferably, hydrophobized silicon dioxides are used which are obtained by silanizing pyrogenic silicon dioxide. Silanized, hydrophobized silicon dioxides are in particular preferably selected from at least one compound from the group formed by trimethylsilylate-coated silicon dioxide, dimethylsilylate-coated silicon dioxide and octylsilylate-coated silicon dioxide.

Preferably, hydrophobized silicon dioxides are used which are obtained by silanizing pyrogenic silicon dioxide.

A large number of suitable hydrophobized silicon dioxides can be obtained commercially. Examples mentioned are Aerosil® R104 V, Aerosil® R106, Aerosil® R202, Aerosil® R805, Aerosil® R812, Aerosil® R812S, Aerosil® R972 and Aerosil® R8200, all from Degussa, and HDK® H2000, HDK® H2050 and HDK® H3004, all from Wacker.

Particularly preferably, the hydrophobized silicon dioxides are used that can be obtained under the names Aerosil® R202, Aerosil® R812S or Aerosil® R972. Very particularly preferably, the silicon dioxide having the INCI name silica silylate is used, which is marketed by Degussa under the name Aerosil® R812S.

Preferred compositions according to the invention are therefore characterized in that they include a hydrophobized silicate, preferably a pyrogenic silicic acid post-treated with polydimethylsiloxane, as the hydrophobized metal oxide powder. Appropriate metal oxides having the INCI name “silica dimethicone silylate” are marketed, for example, by Evonik under the trade name Aerosil® R202. The particle diameter of the primary particles of preferred hydrophobized metal oxides is preferably less than 5 μm, particularly preferably less than 1 μm, and in particular between 1 and 50 nm.

Also preferred are those hydrophobized silicon dioxides that have a BET specific surface area of between 10 and 400 m²/g, preferably between 40 and 300 m²/g and in particular 80 to 150 m²/g.

The powdered compositions according to the invention include an organic polyol as the second essential constituent. Based on their total weight, preferred compositions include the organic polyol in amounts of 12 to 88 wt. %, preferably 15 to 87 wt. %.

The polyol can be used as a single substance or in the form of polyol mixtures. Preferred agents according to the invention are characterized in that they include less than four, preferably one to three, but in particular just one polyol. Polyols from the group of glycerol, 1,2-ethane diol, polyethylene glycols having MW>400, propanediol, butanediol, in particular 1,3-butanediol, hexanediol, in particular 1,6-hexanediol, sorbitol, threitol, erythritol, arabitol, altritol, ribitol, xylitol, galactitol, mannitol, iditol and panthenol are in particular suitable for producing cosmetic compositions. Particularly suitable organic polyols are glycerol, sorbitol and panthenol. These polyols can be converted into a powder form by relatively small amounts of hydrophobized metal oxide powder, generally by less than 10 wt. % metal oxide powder (based on the total weight of the powdered cosmetic composition). Preferred compositions according to the invention are therefore characterized in that they include at least one compound from the group of glycerol, sorbitol and panthenol, preferably glycerol, as the organic polyol.

The weight fraction of the polyol b), based on the total weight of compositions according to the invention, can vary over wide ranges. In compositions according to the invention having a low polyol content, an additional polar solvent is preferably admixed with the compositions for the purpose of balancing, the admixture of water being particularly preferable. Preferred compositions are therefore characterized in that they include, based on their total weight,
12 to 30 wt. %, preferably 15 to 25 wt. %, of organic polyol b) and
40 to 75 wt. %, preferably 50 to 70 wt. % water.

When the polyol is used in a large weight fraction, the admixture of other polar solvents, in particular the admixture of water, is omitted as far as possible. Corresponding compositions preferred according to the invention are characterized in that they include, based on their total weight,
60 to 88 wt. %, preferably 65 to 87 wt. %, of organic polyol b) and
less than 10 wt. %, preferably less than 5 wt. %, preferably less than 1 wt. % water.

The admixture of polyol b) improves the mechanical properties of compositions according to the invention during storage and application, and has advantageous cosmetic effects.

The copolymers c), used as the third essential constituent of compositions according to the invention, are obtained by polymerization of the monomers N-tert-octylacrylamide, acrylic acid, and tert-butylaminoethyl methacrylic acid. At least 90 wt. %, preferably at least 95 wt. %, and in particular at least 97 wt. % of the preferred copolymers c) consists of the monomers N-tert-octylacrylamide, acrylic acid and tert-butylaminoethyl methacrylic acid. Particularly preferred copolymers c) were obtained only from the monomers N-tert-octylacrylamide, acrylic acid and tert-butylaminoethyl methacrylic acid.

The above-described copolymers c) are marketed, for example, under the name Amphomer® (INCI name: octylacrylamide/acrylates/butylaminoethyl methacrylate copolymer) from National Starch.

Preferred compositions according to the invention are characterized in that the weight fraction of the copolymer c) is, based on the total weight of the composition, 0.02 to 12 wt. %, preferably 0.03 to 8.0 wt. %, and in particular 0.05 to 1.0 wt. %.

The composition of some preferred cosmetic agents can be found in the following tables (information in wt. % based on the total weight of the cosmetic agent, unless stated otherwise).

| | Formula 1 | Formula 2 | Formula 3 | Formula 4 | Formula 5 |
|---|---|---|---|---|---|
| Hydrophobized metal | 10 to 30 | 10 to 25 | 10 to 25 | 12 to 22 | 12 to 22 |
| oxide powder | | | | | |
| Organic polyol | 10 to 89 | 12 to 88 | 12 to 88 | 15 to 87 | 15 to 87 |
| Copolymer c) | 0.01 to 15 | 0.02 to 12 | 0.03 to 8.0 | 0.05 to 1.0 | 0.05 to 1.0 |
| Optional additives | Total to 100 | Total to 100 | Total to 100 | Total to 100 | Total to 100 |

| | Formula 6 | Formula 7 | Formula 8 | Formula 9 | Formula 10 |
|---|---|---|---|---|---|
| Silica silylate | 10 to 30 | 10 to 25 | 10 to 25 | 12 to 22 | 12 to 22 |
| Organic polyol | 10 to 89 | 12 to 88 | 12 to 88 | 15 to 87 | 15 to 87 |
| Copolymer c) | 0.01 to 15 | 0.02 to 12 | 0.03 to 8.0 | 0.05 to 1.0 | 0.05 to 1.0 |
| Optional additives | Total to 100 | Total to 100 | Total to 100 | Total to 100 | Total to 100 |

| | Formula 11 | Formula 12 | Formula 13 | Formula 14 | Formula 15 |
|---|---|---|---|---|---|
| Silica silylate | 10 to 30 | 10 to 25 | 10 to 25 | 12 to 22 | 12 to 22 |
| Glycerol | 10 to 89 | 12 to 88 | 12 to 88 | 15 to 87 | 15 to 87 |
| Copolymer c) | 0.01 to 15 | 0.02 to 12 | 0.03 to 8.0 | 0.05 to 1.0 | 0.05 to 1.0 |
| Optional additives | Total to 100 | Total to 100 | Total to 100 | Total to 100 | Total to 100 |

| | Formula 16 | Formula 17 | Formula 18 | Formula 19 | Formula 20 |
|---|---|---|---|---|---|
| Silica dimethicone | 10 to 30 | 10 to 25 | 10 to 25 | 12 to 22 | 12 to 22 |
| Organic polyol | 10 to 89 | 12 to 88 | 12 to 88 | 15 to 87 | 15 to 87 |
| Copolymer c) | 0.01 to 15 | 0.02 to 12 | 0.03 to 8.0 | 0.05 to 1.0 | 0.05 to 1.0 |
| Optional additives | Total to 100 | Total to 100 | Total to 100 | Total to 100 | Total to 100 |

| | Formula 21 | Formula 22 | Formula 23 | Formula 24 | Formula 25 |
|---|---|---|---|---|---|
| Silica dimethicone | 10 to 30 | 10 to 25 | 10 to 25 | 12 to 22 | 12 to 22 |
| Glycerol | 10 to 89 | 12 to 88 | 12 to 88 | 15 to 87 | 15 to 87 |
| Copolymer c) | 0.01 to 15 ‘ | 0.02 to 12 | 0.03 to 8.0 | 0.05 to 1.0 | 0.05 to 1.0 |
| Optional additives | Total to 100 | Total to 100 | Total to 100 | Total to 100 | Total to 100 |

| | Formula 26 | Formula 27 | Formula 28 | Formula 29 | Formula 30 |
|---|---|---|---|---|---|
| Hydrophobized metal | 10 to 30 | 10 to 25 | 10 to 25 | 12 to 22 | 12 to 22 |
| oxide powder | | | | | |
| Organic polyol | 10 to 45 | 12 to 30 | 12 to 30 | 15 to 25 | 15 to 25 |
| Copolymer c) | 0.01 to 15 | 0.02 to 12 | 0.03 to 8.0 | 0.05 to 1.0 | 0.05 to 1.0 |
| Water | 40 to 75 | 40 to 75 | 40 to 75 | 50 to 70 | 50 to 70 |
| Optional additives | Total to 100 | Total to 100 | Total to 100 | Total to 100 | Total to 100 |

| | Formula 31 | Formula 32 | Formula 33 | Formula 34 | Formula 35 |
|---|---|---|---|---|---|
| Silica silylate | 10 to 30 | 10 to 25 | 10 to 25 | 12 to 22 | 12 to 22 |
| Organic polyol | 10 to 45 | 12 to 30 | 12 to 30 | 15 to 25 | 15 to 25 |
| Copolymer c) | 0.01 to 15 | 0.02 to 12 | 0.03 to 8.0 | 0.05 to 1.0 | 0.05 to 1.0 |
| Water | 40 to 75 | 40 to 75 | 40 to 75 | 50 to 70 | 50 to 70 |
| Optional additives | Total to 100 | Total to 100 | Total to 100 | Total to 100 | Total to 100 |

| | Formula 36 | Formula 37 | Formula 38 | Formula 39 | Formula 40 |
|---|---|---|---|---|---|
| Silica silylate | 10 to 30 | 10 to 25 | 10 to 25 | 12 to 22 | 12 to 22 |
| Glycerol | 10 to 45 | 12 to 30 | 12 to 30 | 15 to 25 | 15 to 25 |
| Copolymer c) | 0.01 to 15 | 0.02 to 12 | 0.03 to 8.0 | 0.05 to 1.0 | 0.05 to 1.0 |
| Water | 40 to 75 | 40 to 75 | 40 to 75 | 50 to 70 | 50 to 70 |
| Optional additives | Total to 100 | Total to 100 | Total to 100 | Total to 100 | Total to 100 |

| | Formula 41 | Formula 42 | Formula 43 | Formula 44 | Formula 45 |
|---|---|---|---|---|---|
| Silica dimethicone | 10 to 30 | 10 to 25 | 10 to 25 | 12 to 22 | 12 to 22 |
| Organic polyol | 10 to 45 | 12 to 30 | 12 to 30 | 15 to 25 | 15 to 25 |
| Copolymer c) | 0.01 to 15 | 0.02 to 12 | 0.03 to 8.0 | 0.05 to 1.0 | 0.05 to 1.0 |
| Water | 40 to 75 | 40 to 75 | 40 to 75 | 50 to 70 | 50 to 70 |
| Optional additives | Total to 100 | Total to 100 | Total to 100 | Total to 100 | Total to 100 |

| | Formula 46 | Formula 47 | Formula 48 | Formula 49 | Formula 50 |
|---|---|---|---|---|---|
| Silica dimethicone | 10 to 30 | 10 to 25 | 10 to 25 | 12 to 22 | 12 to 22 |
| Glycerol | 10 to 45 | 12 to 30 | 12 to 30 | 15 to 25 | 15 to 25 |
| Copolymer c) | 0.01 to 15 | 0.02 to 12 | 0.03 to 8.0 | 0.05 to 1.0 | 0.05 to 1.0 |
| Water | 40 to 75 | 40 to 75 | 40 to 75 | 50 to 70 | 50 to 70 |
| Optional additives | Total to 100 | Total to 100 | Total to 100 | Total to 100 | Total to 100 |

| | Formula 51 | Formula 52 | Formula 53 | Formula 54 | Formula 55 |
|---|---|---|---|---|---|
| Hydrophobized metal | 10 to 30 | 10 to 25 | 10 to 25 | 12 to 22 | 12 to 22 |
| oxide powder | | | | | |
| Organic polyol | 10 to 89 | 60 to 88 | 60 to 88 | 65 to 87 | 65 to 87 |
| Copolymer c) | 0.01 to 15 | 0.02 to 12 | 0.03 to 8.0 | 0.05 to 1.0 | 0.05 to 1.0 |
| Water | <10 | <10 | <5.0 | <5.0 | <1.0 |
| Optional additives | Total to 100 | Total to 100 | Total to 100 | Total to 100 | Total to 100 |

| | Formula 56 | Formula 57 | Formula 58 | Formula 59 | Formula 60 |
|---|---|---|---|---|---|
| Silica silylate | 10 to 30 | 10 to 25 | 10 to 25 | 12 to 22 | 12 to 22 |
| Organic polyol | 10 to 89 | 60 to 88 | 60 to 88 | 65 to 87 | 65 to 87 |
| Copolymer c) | 0.01 to 15 | 0.02 to 12 | 0.03 to 8.0 | 0.05 to 1.0 | 0.05 to 1.0 |
| Water | <10 | <10 | <5.0 | <5.0 | <1.0 |
| Optional additives | Total to 100 | Total to 100 | Total to 100 | Total to 100 | Total to 100 |

| | Formula 61 | Formula 62 | Formula 63 | Formula 64 | Formula 65 |
|---|---|---|---|---|---|
| Silica silylate | 10 to 30 | 10 to 25 | 10 to 25 | 12 to 22 | 12 to 22 |
| Glycerol | 10 to 89 | 60 to 88 | 60 to 88 | 65 to 87 | 65 to 87 |
| Copolymer c) | 0.01 to 15 | 0.02 to 12 | 0.03 to 8.0 | 0.05 to 1.0 | 0.05 to 1.0 |
| Water | <10 | <10 | <5.0 | <5.0 | <1.0 |
| Optional additives | Total to 100 | Total to 100 | Total to 100 | Total to 100 | Total to 100 |

| | Formula 66 | Formula 67 | Formula 68 | Formula 69 | Formula 70 |
|---|---|---|---|---|---|
| Hydrophobized metal | 10 to 30 | 10 to 25 | 10 to 25 | 12 to 22 | 12 to 22 |
| oxide powder | | | | | |
| Organic polyol | 10 to 89 | 60 to 88 | 60 to 88 | 65 to 87 | 65 to 87 |
| Copolymer c) | 0.01 to 15 | 0.02 to 12 | 0.03 to 8.0 | 0.05 to 1.0 | 0.05 to 1.0 |
| Water | <10 | <10 | <5.0 | <5.0 | <1.0 |
| Optional additives | Total to 100 | Total to 100 | Total to 100 | Total to 100 | Total to 100 |

| | Formula 71 | Formula 72 | Formula 73 | Formula 74 | Formula 75 |
|---|---|---|---|---|---|
| Silica dimethicone | 10 to 30 | 10 to 25 | 10 to 25 | 12 to 22 | 12 to 22 |
| Glycerol | 10 to 89 | 60 to 88 | 60 to 88 | 65 to 87 | 65 to 87 |
| Copolymer c) | 0.01 to 15 | 0.02 to 12 | 0.03 to 8.0 | 0.05 to 1.0 | 0.05 to 1.0 |
| Water | <10 | <10 | <5.0 | <5.0 | <1.0 |
| Optional additives | Total to 100 | Total to 100 | Total to 100 | Total to 100 | Total to 100 |

The cosmetic compositions according to the invention can include other auxiliary substances, care substances and additives. The weight fraction of the other ingredients included in the powdered compositions according to the invention in addition to components a) to c), in particular of the auxiliary substances, care substances and additives included in said compositions based on the total weight of the powdered compositions according to the invention is preferably less than 10 wt. %, preferably less than 5.0 wt. %, particularly preferably less than 2.0 wt. %, and in particular less than 1.0 wt. %. The weight fraction of these auxiliary substances, care substances and additives based on the total weight of the cosmetic agents according to the invention can for example be 0.001 to 2 wt. %, in particular 0.01 to 0.5 wt. %.

The powdered compositions according to the invention can be provided in any containers, provided that the powder is never liquefied upon mechanical loading when the composition is removed. For example, pots, bottles or Tetra Paks are suitable, it being possible to design the container to have a pouring or dispensing device for example.

The present application also relates to the use of a cosmetic composition according to the invention for temporarily deforming keratin-containing fibers, in particular human hair. When using the powdered composition for temporarily deforming keratin fibers, the desired amount of the powdered composition is preferably removed from the container first of all. The composition can be provided either directly onto the keratin fiber to be treated or, for example, onto the hand. In the first case, the applied powder can be subjected to mechanical loading, for example by means of the hands, directly on the keratin fiber, as a result of which the liquid, aqueous phase is released directly onto the fibers. If the powdered composition is first provided onto the hand, it can firstly be carefully distributed through the hair and only then mechanically loaded more strongly, for example by massaging the powder into the hair in a targeted manner. As a result, the liquid, aqueous phase is released on the hair.

Of course, it is also possible to already rub the powdered composition when it is on the hand and to then apply the resultant liquid or paste-like agent to the keratin fibers. This approach is, however, not preferable since it dispenses with a significant advantage of the powdered consistency of the styling agent, i.e. the ease of distribution. Of course, the powdered composition can also be applied using an aid, for example an applicator brush, a sponge, a towel, a brush or a comb.

The present application also relates to a method for temporarily deforming keratin-containing fibers, in particular human hair, in which a cosmetic composition according to the invention is applied to the keratin fibers and these are temporarily fixed in terms of their shape, characterized in that a plastically deformable compound is formed from the cosmetic composition before, during or after application thereof to the keratin fibers, by the application of a force.

### EXAMPLES

The powdered styling agents V1 to V6 were produced as described below (information in wt. %):

| | V1 | V2 | V3 | V4 | V5 | V6 |
|---|---|---|---|---|---|---|
| Silica silylate | 15 | 17 | 17 | 15 | 15 | 15 |
| Glycerol | Total | 20 | 20 | Total | Total | Total |
| | to | | | to | to | to |
| | 100 | | | 100 | 100 | 100 |
| Copolymer c) | 0.05 | 0.05 | 0.05 | 0.05 | 0.05 | 0.05 |
| Water | — | Total | Total | — | — | — |
| | | to | to | | | |
| | | 100 | 100 | | | |

All the constituents were mixed in a vessel, apart from silica silylate. This liquid was mixed with the hydrophobized silicon dioxide powder silica silylate. After stirring for 30 to 45 seconds, a stable powder had formed in each case. The styling powder thus obtained was poured into polyethylene bottles.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A powdered cosmetic composition, comprising:
a) 10 to 30 wt. % of a hydrophobized metal oxide powder
b) 10 to 89 wt. % of an organic polyol
c) 0.01 to 15 wt. % of at least one copolymer from the monomers
b1) N-tert-octylacrylamide
b2) acrylic acid
b3) tert-butylaminoethyl methacrylic acid
b4) and possibly other monomers.

2. The composition according to claim 1, wherein the composition comprises 10 to 25 wt % hydrophobized metal oxide powder.

3. The composition according to claim 1, wherein the composition comprises 12 to 22 wt. % hydrophobized metal oxide powder.

4. The composition according to claim 1, wherein the hydrophobized metal oxide powder includes a hydrophobized silicate obtained by silanizing pyrogenic silicon dioxide.

5. The composition according claim 1, wherein the organic polyol comprises 12 to 88 wt. % of the total composition.

6. The composition according to claim 1, wherein the organic polyol comprises 15 to 87 wt. % of the total composition..

7. The composition according to claim 1, wherein the composition comprises, based on its total weight, 12 to 30 wt. % organic polyol and 40 to 75 wt % water.

8. The composition according to claim 1, wherein the composition comprises, based on its total weight, 15 to 25 wt. % of organic polyol b) and 50 to 70 wt. %, water.

9. The composition according to claim 1, wherein the composition comprises, based on its total weight, 60 to 88 wt. % organic polyol b) and less than 10 wt % water.

10. The composition according to claim 1, wherein the composition comprises, based on its total weight, 65 to 87 wt. % of organic polyol b) and less than 5 wt. % water.

11. The composition according to claim 1, wherein the organic polyol b) includes glycerol.

12. The composition according to claim 1, wherein the composition comprises 0.02 to 12 wt % of copolymer c).

13. The composition according to claim 1, wherein the composition comprises 0.03 to 8.0 wt. %, of copolymer c).

14. The composition according to claim 1, wherein the composition comprises 0.05 to 1.0 wt. % of copolymer c).

15. A method for temporarily deforming keratin-containing fibers, in which a cosmetic composition according to claim 1 is applied to the keratin fibers and the keratin-containing fibers are temporarily fixed in terms of their shape, wherein a plastically deformable compound is formed from the cosmetic composition before, during or after application thereof to the keratin fibers, by the application of a force.
